(19) United States
(12) Patent Application Publication
(10) Pub. No.: US 2012/0100072 A1
(43) Pub. Date: Apr. 26, 2012

(54) PET IMAGING OF FIBROGENESIS

(75) Inventors: Salah Chettibi, Amersham (GB); Ben Newton, Amersham (GB); Magne Solbakken, Oslo (NO)

(73) Assignee: GE HEALTHCARE LIMITED, LITTLE CHALFONT (GB)

(21) Appl. No.: 13/376,944

(22) PCT Filed: Jun. 10, 2010
(86) PCT No.: PCT/EP10/58135

§ 371 Date: Dec. 8, 2011

Related U.S. Application Data
(60) Provisional application No. 61/185,669, filed on Jun. 10, 2009.

(30) Foreign Application Priority Data
Jun. 10, 2009 (GB) 0910013.2

(51) Int. Cl. A61K 51/00

(52) U.S. Cl. 424/1.69

(57) ABSTRACT

The present invention relates to peptide compounds and their use for in vivo imaging using positron emission tomography (PET). More specifically, the invention relates to the use of such peptide-based compounds in a method for the in vivo imaging of liver fibrosis.

### CROSS-REFERENCE TO RELATED APPLICATIONS

This application is a filing under 35 U.S.C. §371 and claims priority to international patent application number PCT/EP2010/058135 filed Jun. 10, 2010, published on Dec. 16, 2010 as WO 2010/142754, which claims priority to application number 0910013.2 filed in Great Britain on Jun. 10, 2009 and application No. 61/185,669 filed in the United States on Jun. 10, 2009.

### FIELD OF THE INVENTION

The present invention relates to peptide compounds and their use for in vivo imaging using positron emission tomography (PET). More specifically, the invention relates to the use of such peptide-based compounds in a method for the in vivo imaging of liver fibrosis.

### SUMMARY OF THE INVENTION

The in vivo imaging technique positron emission tomography (PET) provides excellent sensitivity and resolution, so that even relatively small changes in a lesion can be observed over time. A preferred positron-emitting nuclide for use in PET is ¹⁸F, which has a half-life of approximately 110 minutes, decays 97% by positron emission, and has a maximum energy of 0.69 MeV. An ¹⁸F-labelled peptide that targets a biomarker representative of a particular disease state can be used in the detection and characterisation of that disease state.

Hepatic stellate cells (HSC) are widely regarded as the principal fibrocompetent cell in the liver (Bedossa and Paradis J. Pathol. 2003; 200: 504-515). During progressive liver fibrosis, HSC activate and proliferate, but during resolution of fibrosis there is extensive HSC apoptosis that coincides with degradation of the liver scar. This progressive stage represents the early stage of fibrosis, and is termed “fibrogenesis”. Activated HSC associated with fibrogenesis have upregulated expression of the integrin αᵥβ₃ (Zhou et al J. Biol. Chem. 2004; 279(23): 23996-24006). αᵥβ₃ therefore presents itself as a potential biomarker for in vivo imaging of liver fibrogenesis.

Until now, the primary focus of attention for αᵥβ₃-targeting PET tracers has been the in the diagnosis of angiogenesis-related diseases, primarily tumours, and in particular metastatic tumours. For example, WO 2005/012335 teaches ¹⁸F-labelled peptide-based in vivo imaging agents comprising the arginine-glycine-aspartic acid (RGD) motif that bind to the integrin αᵥβ₃ that are useful in the in vivo diagnosis or imaging of a disease or condition associated with angiogenesis. WO 2006/030291 also teaches particular ¹⁸F-labelled RGD peptide-based compounds that are useful for in vivo imaging of angiogenesis-related diseases and conditions. It has also been suggested that RGD peptide-based in vivo imaging agents are useful for in vivo imaging and diagnosis of disease conditions associated with collagen deposition, including liver fibrosis (WO 2006/054904). WO 2006/054904 discloses a range of in vivo imaging moieties, including ¹⁸F. However, in vivo data that has been presented more recently demonstrates that ¹⁸F-labelled RGD peptides have characteristics rendering them unsuitable for optimum in vivo imaging of the liver. A report on the biodistribution of an ¹⁸F-labelled RGD peptide comprising a PEG linker in healthy human volunteers demonstrated a mean initial uptake in the liver of around 15%, decreasing to around 8% after 4 hours post injection (M c Parland et al J. Nuc. Med. 2008; 49(10): 1664-7). The same ¹⁸F tracer was evaluated for its ability to detect tumours in metastatic breast cancer patients (Kenny et al J. Nuc. Med. 2008; 49: 879-886). In this study, background uptake in the liver was so high that liver metastases appeared as hypointense foci.

Nonalcoholic fatty liver disease (NAFLD) refers to a wide spectrum of liver disease ranging from simple fatty liver (steatosis), to nonalcoholic steatohepatitis (NASH), to cirrhosis (irreversible, advanced scarring of the liver). Around 24% of the US population is thought to have NAFLD, which progresses to NASH at low frequency. NAFLD is associated with metabolic syndrome, which is linked with obesity, hyperlipidemia, hypertension and type II diabetes. It is believed that in the region of 47 million individuals in USA have metabolic syndrome. An estimated 8.6 million of the US population are thought to have NASH, which may become associated with fibrosis and cirrhosis with 20-28% of patients with NASH developing into cirrhosis over a decade. NAFLD is therefore very common and represents the less severe end of a spectrum that may progress to NASH, and ultimately to cirrhosis of the liver. Liver fibrosis is an indicator of a risk of progression from NASH to cirrhosis.

Currently-used approaches for the detection of liver fibrosis have some notable disadvantages. Liver biopsy analysed histologically for the pattern of collagen deposition is considered the gold standard for assessing liver disease stage and liver fibrosis. However, the procedure is associated with some morbidity, occasional mortality, high costs, sampling errors, and high inter-observer variability among hepatopathologists in categorising the degree of fibrosis. Errors in stage diagnosis can be made because biopsy sampling of liver results in only 1/50,000^{th} of the liver being assessed. Furthermore, in order to monitor disease progression in a timely manner, it is recommended that repeat biopsies are carried out every 3-5 years. Less invasive strategies are available, such as blood tests, but current blood tests for detecting liver fibrosis are of limited value clinically as they cannot be used for assessing the degree of fibrosis or for discriminating fibrosis from cirrhosis. At present, there is no method that can distinguish NAFLD from NASH, or satisfactorily quantify and characterise fibrosis in NASH. There is therefore no means by which liver fibrosis can effectively be characterised and monitored via a non-invasive procedure. This has a negative impact on the provision of early therapeutic intervention, which may slow or halt liver fibrosis. An in vivo imaging method capable of detecting the early stages of fibrosis would be useful in the clinical management of the NAFLD disease process.

The invention is useful for assessment of the presence, location and/or amount of activated HSC, providing an indicator of fibrogenesis. This is particularly advantageous because fibrogenic tissue is a better marker of early active disease than fibrotic tissue, the latter also being present where the disease process is resolving. Identification of the disease process can therefore be done at a stage when implementation of treatment can be most efficacious.

### BRIEF DESCRIPTION OF THE FIGURES

FIGS. 1 and 2 demonstrate the results of a PET imaging study in an animal model of liver fibrogenesis.

FIG. 1 illustrates % ID/cc (percentage injected dose per cubic centimetre of tissue) in rat livers and reference tissue (muscle) at different days post-bile duct ligation (BDL) or sham surgery. All imaging data was taken from 60-90 minutes post intravenous injection of PET Tracer 1.

FIG. 2 illustrates representative co-registered PET-CT (positron-emission tomography-computed tomography) images showing the uptake of PET Tracer 1 in the BDL (top row) and sham operated (bottom row) rat liver (for reference marked “L” in far left images) and kidneys (for reference marked “K” in far left images), normalised for injected dose, between days 2 and 30 post initiation of the surgery.

The figures clearly show a significant difference in the uptake of PET Tracer 1 in the liver of the BDL animals in comparison to the sham-operated animals.

### DETAILED DESCRIPTION OF THE INVENTION

In one aspect, the present invention relates to a method to determine the presence, location, and/or amount of fibrogenic tissue in the liver of a subject, said method comprising the following steps:

(i) administering a detectable quantity of a positron emission tomography (PET) tracer of Formula I to said subject;
(ii) allowing the administered PET tracer of step (i) to bind to any fibrogenic tissue in said liver;
(iii) detecting signals emitted by the bound PET tracer of step (ii) by PET; and,
(iv) generating an image representative of the location and/or amount of said signals;
wherein said PET tracer is of Formula I:

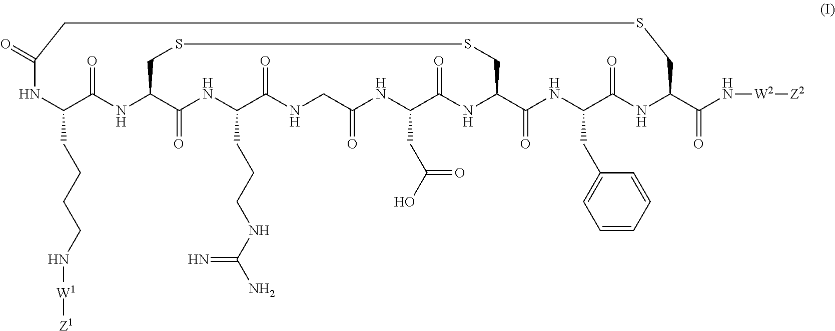

wherein:
one of Z¹ and Z² is a group comprising ¹⁸F, and the other of Z¹ and Z² is hydrogen; and,
each of W¹ and W² is independently a bivalent linker moiety of Formula Ia:

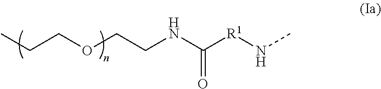

wherein
n is an integer from 1 to 10;
R¹ is C₁₋₅ alkylene, C₂₋₅ oxoalkylene, C₁₋₅ oxaalkylene, or is a C₂₋₅ carbonyl-substituted oxaalkylene; and,
the dotted line represents the point of attachment to either Z¹ or Z².

The term “fibrogenic tissue” as used herein specifically relates to tissue wherein fibrogenesis is taking place. The term “fibrogenesis” relates to the active, progressive stage of fibrosis, when, amongst other things, hepatic stellate cells (HSC) are activated and express integrins. HSC are widely regarded as the principal fibrocompetent cell in the liver. During fibrogenesis, HSC activate and proliferate, but during resolution of fibrosis there is extensive HSC apoptosis that coincides with degradation of the liver scar. Furthermore, during fibrogenesis, the deposition of extracellular matrix (ECM) components, such as collagen, has not yet taken place. The presence of ECM components is therefore characteristic of the later stages of fibrosis and of resolution of fibrosis. Targeting the disease process during fibrogenesis therefore provides a better indication of active disease where application of treatment is most appropriate.

The “subject” of the invention is an animal having a liver. The term “liver” is to be understood in the well-known physiological sense, i.e. a vital organ present in vertebrates and some other animals; having a wide range of functions, including detoxification, protein synthesis, and production of biochemicals necessary for digestion. In a preferred embodiment, said subject is an intact mammalian body in vivo, and most preferably an intact human body in vivo.

The step of “administering” a detectable quantity of the PET tracer of Formula I to a subject can be understood as a method that results in the systemic presence of said PET tracer within said subject. Administering is preferably carried out parenterally, and most preferably intravenously. The intravenous route represents the most efficient way to deliver the PET tracer throughout the body of the subject, and also does not represent a substantial physical intervention on the body of the subject. By the term “substantial” is meant an intervention which requires professional medical expertise to be carried out, or which entails a substantial health risk even when carried out with the required professional care and expertise. The PET tracer is preferably administered as a radiopharmaceutical composition, as defined herein. The method of the invention can also be understood as comprising the above-defined steps (ii)-(iv) carried out on a subject to whom the PET tracer has been pre-administered.

A “detectable quantity” of the PET tracer of Formula I means an amount of said PET tracer that is sufficient to yield a signal detectable by PET. For example, typical radionuclide dosages of 0.037 MBq to 3.70 GBq (0.01 to 100 mCi), preferably 3.7 MBq to 1.85 GBq (0.1 to 50 mCi), most preferably 37 to 740 MBq to (1 to 20 mCi), will normally be sufficient per 70 kg bodyweight.

The term “PET tracer” in the context of the present invention refers to a compound comprising a positron-emitting radioactive isotope. Such a PET tracer is designed to bind to a particular cell component, e.g. a cell surface receptor, such that detection of signals emitted by the positron-emitting radioactive isotope indicates the location and quantity of that cell component.

The step of “allowing” the PET tracer to bind to any fibrogenic tissue in the liver of said subject follows the administering step and precedes the detecting step. What in effect takes place during said allowing step is that the PET tracer moves dynamically within the system of said subject and come into contact with various tissues therein. It is crucial for the success of the method of the invention that the time period for the allowing step is selected to enable specific interaction to take place between the PET tracer and any fibrogenic cells in the liver, and preferably also for at least a proportion of non-specifically bound PET tracer to have moved away from the liver. A certain point in time will be reached when detection of PET tracer specifically bound to any fibrogenic cells in the liver is enabled as a result of the ratio between PET tracer bound to said fibrogenic cells versus that bound to non-fibrogenic cells. An ideal such ratio is at least 2:1.

The “detecting” step is then carried out by placing the subject in a PET scanner to detect pairs of annihilation photons produced when positrons emitted by ¹⁸F travel up to a few millimeters, and encounter and annihilate an electron. These annihilation photons are the “signals” emitted by the PET tracer.

The “generating” step of the method of the invention is carried out by a computer which applies a reconstruction algorithm to the detected signals to yield a dataset. This dataset is then manipulated to generate an image showing of the liver of the subject. The image obtained will be representative of the presence, location, and/or amount of fibrogenic tissue in the liver of the subject.

A “group comprising ¹⁸F” can signify ¹⁸F per se, or a chemical group that includes ¹⁸F. Suitably, for said PET tracer of Formula I, said group comprising F is a chemical ¹⁸F group that does not undergo facile metabolism in blood. That is because such metabolism would result in the ¹⁸F being cleaved off the PET tracer before the PET tracer reaches the desired in vivo targeting site, i.e. the liver. For example, ¹⁸F may form part of a [¹⁸F]fluoroalkyl or [¹⁸F]fluoroalkoxy group, since alkyl fluorides are resistant to in vivo metabolism. Alternatively, ¹⁸F may be attached via a direct covalent bond to an aromatic ring.

The term “alkylene” means a bivalent chain of —CH₂— groups, wherein the number of —CH₂— groups is between 1 and 5.

The term “oxoalkylene” refers to an alkylene as defined above that further comprises at least one carbonyl group in the chain. The term “carbonyl” refers to the group —C(═O)—. A chain wherein two or more carbonyl groups are linked together is not encompassed; the skilled person would understand that such an arrangement is not chemically feasible.

The term “oxaalkylene” refers to an alkylene as defined above that further comprises at least one oxygen atom in the chain, i.e. the group —O—. A chain wherein two or more oxygen atoms are linked together (—O—O—) is not encompassed; the skilled person would understand that such groups are highly unstable and therefore not suitable in the context of the present invention. Preferably, the oxygen atom is present as an ether linkage, i.e. —C—O—C—.

The term “C₂₋₅ carbonyl-substituted oxaalkylene” refers to an oxaalkylene as defined above that further comprises a carbonyl group in the chain, wherein carbonyl is as defined above. Encompassed in this definition are ester linkages, wherein the term “ester linkage” refers to —C(═O)—O—. Also encompassed are groups such as —C(═O)—CH₂—O—, —C(═O)—CH₂— CH₂—O— and —C(═O)—CH₂—O—CH₂—. Specifically excluded are reactive groups such as acid anhydride, i.e. —C(═O)—O—C(═O)—. The skilled person would understand that such reactive groups are not suitable in the context of the present invention.

The peptide portion of the PET tracer of Formula I may be prepared by standard methods of peptide synthesis, for example, solid-phase peptide synthesis, for example, as described in Atherton, E. and Sheppard, R. C.; “Solid Phase Synthesis”; IRL Press: Oxford, 1989. Incorporation of the aminoxy group may be achieved by formation of a stable amide bond formed by reaction of a peptide amine function with an activated acid and introduced either during or following the peptide synthesis. The reader is referred to Indrevoll et al (Bioorg. Med. Chem. Lett. 2006; 16: 6190-3) for a more detailed description of how to obtain the peptide portion of the PET tracer of Formula I.

Addition of the ¹⁸F label can be carried out by techniques well-known in the art of radiochemistry. For example, ¹⁸F can be introduced by N-alkylation of amine precursor compounds with a labeling compound such as ¹⁸F(CH₂)₃OMs (where OMs is mesylate) to give N—(CH₂)₃¹⁸F. Primary amine-containing precursor compounds can also be labelled with ¹⁸F by reductive amination using the labeling compound ¹⁸F—C₆H₄—CHO, as taught by Kahn et al (J. Lab. Comp. Radiopharm. 2002; 45: 1045-1053) and Borch et al (J. Am. Chem. Soc. 1971; 93: 2897). This approach can also be applied to aminoxy derivatives of peptides as taught by Poethko et al (J. Nuc. Med., 2004; 45: 892-902).

Amine-containing precursor compounds can also be labelled with ¹⁸F by reaction with an ¹⁸F-labelled active ester labeling compound such as:

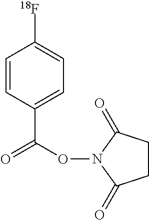

to give amide bond linked products. The N-hydroxysuccinimide ester shown above and its use to label peptides is taught by Vaidyanathan et al (Nucl. Med. Biol., 1992; 19(3): 275-281) and Johnstrom et al (Clin. Sci., 2002; 103 (Suppl. 48): 45-85). Further details of synthetic routes to ¹⁸F-labelled derivatives are described by Bolton (J. Lab. Comp. Radiopharm., 2002; 45: 485-528).

Reference is also made to WO 03/006491, WO 2005/012335, and WO 2006/030291 which describe synthesis of peptides analogous to those of the present invention.

Any of the above-described methods for incorporating ¹⁸F may be applied in the preparation of a PET tracer of Formula I from the corresponding precursor compound of Formula II:

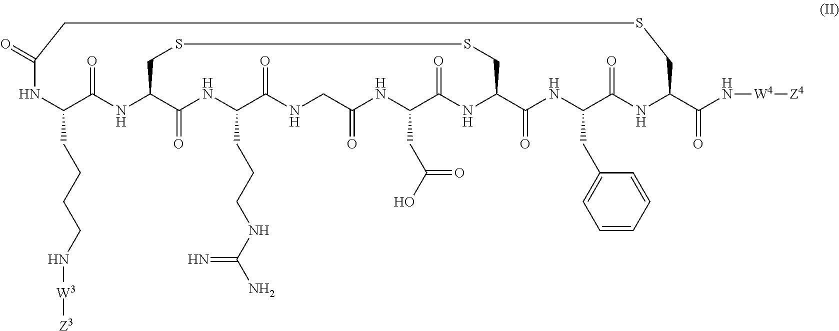

wherein W³ and W⁴ are as defined above for W¹ and W², respectively, of Formula I; and,
Z³ and Z⁴ are both hydrogen.

A preferred ¹⁸F labelling compound of the present invention is of Formula IIa:

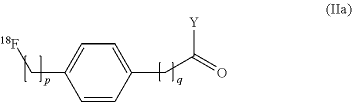

wherein:
p is an integer of 0 to 20;
q is an integer of 0 to 10; and,
Y is hydrogen, C₁₋₆ alkyl (such as methyl), or phenyl.

Therefore, in a preferred embodiment, said group comprising ¹⁸F of Formula I is an aromatic group, and is most preferably [¹⁸F]fluorophenyl. A preferred location on Formula I for the group comprising ¹⁸F is at Z¹.

The labelling compound of Formula IIa may be prepared from the corresponding starting compound of Formula IIb:

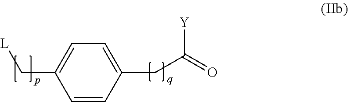

or a protected derivative thereof, wherein L is a leaving group; preferably when p≧1, L is p-toluenesulphonate, trifluoromethanesulphonate, or methanesulphonate or a halide, and when p is 0 L is p-trialkyl ammonium salt or p-nitro; and, Y and q are as described above for the labelling compound of Formula IIa. The starting compound of Formula lib is reacted with cyclotron produced aqueous [¹⁸F]-fluoride, suitably pre-activated by evaporation from a base (for example, from tetrabutylammonium or K₂CO₃/Kryptofix-222), in a suitable solvent such as acetonitrile, N,N-dimethylformamide, or dimethyl sulphoxide, typically at ambient or at elevated temperature, for example up to 140° C. The aldehyde or ketone function of compounds of Formula IIa can also be rapidly generated from their protected versions such as acetals or ketals by simple acid treatment following radiofluorination.

The PET tracer of Formula I may be prepared by means of a kit, e.g. comprising a precursor compound of Formula II and a labelling compound of Formula IIa. In use of the kits, the labelling compound of Formula IIa would be added to the precursor compound of Formula II, which may suitably be dissolved in aqueous buffer (pH 1-11). After reaction at a non-extreme temperature for 1 to 70 minutes, the labelled peptide may be purified, for example, by solid-phase extraction (SPE) or high performance liquid chromatography (HPLC) and collected.

The nature of the bivalent linker moiety W¹ or W² can also be used to modify the biodistribution of the PET tracer of Formula I. Thus, e.g. ether groups in the linker will help to minimise plasma protein binding. When the bivalent linker moiety comprises a polyethyleneglycol (PEG) building block, the linker group may function to modify the pharmacokinetics and blood clearance rates of the PET tracer in vivo. Such “biomodifier” linker groups may accelerate the clearance of the imaging agent from background tissue, such as muscle or liver, and/or from the blood, thus giving a better diagnostic image due to less background interference. A biomodifier linker group may also be used to favour a particular route of excretion, e.g. via the kidneys as opposed to via the liver.

In the PET tracer of Formula I, it is preferred that n of the bivalent linker moiety of Formula Ia is from 3 to 5. For W¹ a preferred n is 5, and for W² a preferred n is 3. For W′, R¹ is preferably a C₁₋₅ alkoxyalkenyl, most preferably a C₁₋₃ alkoxyalkenyl, and especially preferably is —CH₂—O—. For W², R¹ is preferably C₂₋₅ carboxyalkoxyalkenyl, most preferably C₂₋₄ carboxyalkoxyalkenyl, and especially preferably —CH₂—O—CH₂—C(═O)—.

An example of a preferred PET tracer for use in the method of the invention is:

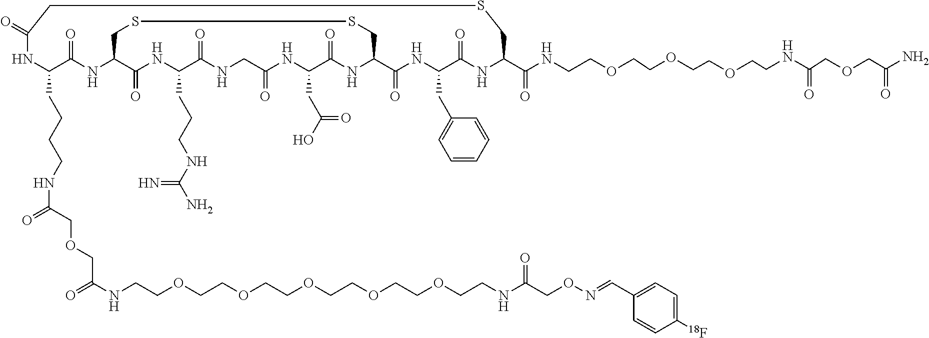

The above PET tracer is referred to herein as “PET tracer 1”, and may be obtained by the method described by Kenny et al (J. Nuc. Med. 2008; 49: 879-86). PET Tracer 1 has been analysed both in vitro and in vivo (as described in Examples 1-3 below), and a significant difference was found in the uptake of PET Tracer 1 in an animal model of liver fibrogenesis in comparison to the corresponding negative control animal model, suggesting that this PET Tracer is capable of imaging fibrogenesis.

The method of the invention is preferably carried out wherein said PET tracer is provided as a radiopharmaceutical composition. A “radiopharmaceutical composition” is defined in the present invention as a composition comprising a PET tracer of Formula I together with a biocompatible carrier in a form suitable for mammalian administration. The “biocompatible carrier” is a fluid, especially a liquid, in which the PET tracer of Formula I is suspended or dissolved, such that the composition is physiologically tolerable, i.e. can be administered to the mammalian body without toxicity or undue discomfort. The biocompatible carrier is suitably an injectable carrier liquid such as sterile, pyrogen-free water for injection; an aqueous solution such as saline (which may advantageously be balanced so that the final product for injection is either isotonic or not hypotonic); an aqueous solution of one or more tonicity-adjusting substances (e.g. salts of plasma cations with biocompatible counterions), sugars (e.g. glucose or sucrose), sugar alcohols (e.g. sorbitol or mannitol), glycols (e.g. glycerol), or other non-ionic polyol materials (e.g. polyethyleneglycols, propylene glycols and the like). The biocompatible carrier may also comprise biocompatible organic solvents such as ethanol. Such organic solvents are useful to solubilise more lipophilic compounds or formulations. Preferably the biocompatible carrier is pyrogen-free water for injection, isotonic saline or an aqueous ethanol solution. The pH of the biocompatible carrier for intravenous injection is suitably in the range 4.0 to 10.5. The radiopharmaceutical composition may optionally contain further ingredients such as buffers, pharmaceutically acceptable solubilisers (for example cyclodextrins or surfactants such as Pluronic, Tween, or phospholipids), pharmaceutically acceptable stabilisers or antioxidants (such as ascorbic acid, gentisic acid or para-aminobenzoic acid) or bulking agents for lyophilisation (such as sodium chloride or mannitol).

The radiopharmaceutical composition may be prepared as described above for the PET tracer, but under aseptic manufacture conditions to give the desired sterile product. The radiopharmaceutical composition may alternatively be prepared under non-sterile conditions, followed by terminal sterilisation using e.g. gamma-irradiation, autoclaving, dry heat or chemical treatment (e.g. with ethylene oxide).

In an alternative embodiment, the method of the invention may be used to monitor the progression of fibrogenesis within said subject. In this case, the method of the invention is carried out at two separate points in time. For example, the method may be carried out wherein the interval between the two separate points in time is in the range 1-6 years, preferably 3-5 years. In the interval between the two separated time points, an antifibrogenic treatment may be applied to the subject. In this way, evaluation of the effectiveness of an antifibrogenic treatment may be carried out. Examples of known drug treatments for the inhibition of fibrogenesis include essential phospholipids (EPL), silymarin and ursodeoxycholic acid (UDCA) (see Chapter 31 “Hepatology” by Hanz-Dieter Kuntz, Birkhäuser, 2006, and in particular page 587 discussion of adjuvant therapy). Polyenephosphatidylcholine (PPC), a major component of EPL, prevented alcoholic liver fibrosis in baboons (J. Hepatol. 2009; 50(6): 1236-46). A study by Lieber et al (J. Clin. Gastroenterol. 2003; 37(4): 336-9) reports that silymarin retards the progression of alcohol-induced hepatic fibrosis in baboons. UDCA is an approved treatment for primary biliary cirrhosis (PBC). It has been disclosed that UDCA therapy significantly delays the progression of liver fibrosis in PBC (Corpechot et al Hepatology 2001; 32(6): 1196-9).

In an additional aspect, the present invention provides a method of diagnosis comprising the method of the invention as suitably and preferably defined herein and further comprising the additional step of (v) attributing the location and/or amount of signals to a particular clinical picture. Specifically, there is a direct correlation between the amount of signals and the extent of fibrogenesis.

In other aspects, the present invention provides the PET tracer of Formula I, as defined herein, for use in the method of the invention, or in the method of diagnosis of the invention, as defined herein. For this aspect of the invention, the PET tracer, and preferred embodiments thereof, are as defined above for the method of the invention.

In yet further aspects, the present invention provides the PET tracer of Formula I, as defined herein, in the manufacture of a medicament for carrying out the method of the invention, or in the method of diagnosis of the invention, defined herein. For this aspect of the invention, the PET tracer, and preferred embodiments thereof, are as defined above for the method of the invention.

### BRIEF DESCRIPTION OF THE EXAMPLES

Example 1 describes an in vitro assay used to evaluate binding to membranes prepared from EA-Hy926 cells.
Example 2 describes an in vivo model of liver fibrogenesis, the bile duct ligation (BDL) model, as well as the corresponding negative control model or “sham animal”.
Example 3 describes the longitudinal imaging studies that were carried out with PET Tracer 1.

### LIST OF ABBREVIATIONS USED IN THE EXAMPLES

° C. degrees Celsius
μm micrometre(s)
% ID/cc percentage of the injected dose per cubic centimetre of tissue
BDL bile duct ligation
cm³ centimetre(s) cubed
CT computed tomography
g gram(s)
i.d. injected dose
i.v. intravenous(ly)
keV kilo electronvolt(s)
kVp kilovolt peak
MBq mega Becquerel(s)
mg/kg milligram(s) per kilogram
ml millilitre(s)
mm millimetre(s)
nM nanomolar
nsec nanosecond(s)
PET positron emission tomography
ROI(s) region(s) of interest
s.c subcutaneously

### EXAMPLES

### Example 1

### Binding to Membranes Prepared from EA-Hy926 Cells

The inhibition constant was measured using a previously-described membrane binding assay (Indrevoll et al, Bioorg & Med Chem Lett, 2006, 16, 6190-6193).
In brief, membranes from the human endothelial adenocarcinoma cell line EA-Hy926 were prepared and the K_{d} calculated for the purified membrane fraction. A competitive binding assay was then established to measure inhibition constants. ¹²⁵I-echistatin (GE Healthcare; Code IM304) was used as the labelled ligand and cold echistatin as a reference standard.
A total of sixteen dilutions of cold test compound (either cold echistatin or cold PET tracer) were prepared and mixed with a combination of ¹²⁵I-echistatin and membrane prior to incubation for 1 hour at 37° C. Following several washes, the bound material was harvested on a filter using a Skatron micro harvester. The filterspots were finally excised and counted in a Packard γ-counter.
PET tracer 1 (prepared by the method described by Kenny et al, J. Nuc. Med. 2008; 49: 879-86), when assessed with the above-described assay, demonstrated an affinity of 5-10 nM.

### Example 2

### Bile Duct Ligation (BDL) and Sham Animals

### 2(i) Animal Model Set-Up

Outbred male Sprague Dawley rats (180-200 g; Charles River) were used in all bile duct ligation (BDL) and sham studies. After 6 days acclimatization rats were divided into 2 groups (BDL group and sham group).
For the BDL animals, the abdomen was shaved and swabbed with betadine solution followed by 5 mg/kg carprofen subcutaneously (s.c.) and 5 mg/kg bupronorphine s.c. and under Isoflurane anaesthesia a mid-line laparotomy was performed and the common bile duct located. Bile duct was double ligated, the first ligation made between the junction of the hepatic ducts and the second above the entrance of the pancreatic ducts.
The second group (sham animals) abdomen was shaved and swabbed with betadine solution followed by 5 mg/kg carprofen s.c. and 5 mg/kg bupronorphine s.c. Animals underwent sham surgery where bile duct was manipulated and a suture passed under the bile duct.
Before closing 2-3 ml saline was administered into the peritoneum of each animal. Fascia and skin were closed and animals administered with 2 mg/kg metaclopromide s.c, 5 mg/kg Baytril s.c., and ˜2 ml saline s.c. Carprofen was given (5 mg/kg) as required over the next couple of days. Animals were closely monitored for the duration of the experiment.

### 2(ii) Administration of Test Compound and Biodistribution

On the appropriate day post surgery, BDL and sham animals were removed and put under isoflurane anaesthesia, then each animal was injected with 0.3 ml intravenously (i.v.) via tail vein (˜3 MBq). At the appropriate time point post-injection of the test item, each animal was re-anaesthetised with isoflurane, sacrificed by cervical dislocation, weighed, and the weight recorded via a barcode scanning system. Each animal was dissected and the following organs and tissues were removed and counted using BASIL counter protocol 40 or manual counting: bone*; muscle*; blood*; kidneys; bladder & urine (B/U); lung; liver*; spleen; stomach & contents; small and large intestine (SI & LI); heart; thyroid; skin*; carcass; injection site; (* weighed samples).
The recorded activity in a whole organ (e.g., liver) was corrected for background radioactivity and for radioactive decay and the biodistribution of radioactivity calculated by reference to Formula 1:

$$\% i . d . Organ = \frac{A}{B} \times 100$$

where:
A=counts per second measured in organ
B=total counts per second measured in all samples (excluding the injection site)
The percentage of injected radioactivity in the weighed tissue samples was calculated to give % i.d. in the entire tissue by reference to Formula 2:

$$\% i . d . tissue = \frac{( {Z}_{s} \cdot {W}_{b} \times F ) / B}{{W}_{s}} \times 100$$

where:
Zₛ=counts per second in sample
Wₛ=weight of sample in grams
W_{b}=weight of animal in grams immediately after sacrifice
B=total counts per second measured in all the samples (excluding the injection site)
F=tissue specific factor representing the mass of the tissue as a proportion of the total body weight of the animal

| Tissue | F |
|---|---|
| Bone | 0.05 |
| Muscle | 0.43 |
| Blood | 0.058 |
| Skin | 0.18 |
| Fat | 0.07 |

### Example 3

### Longitudinal Imaging Studies of PET Tracer 1

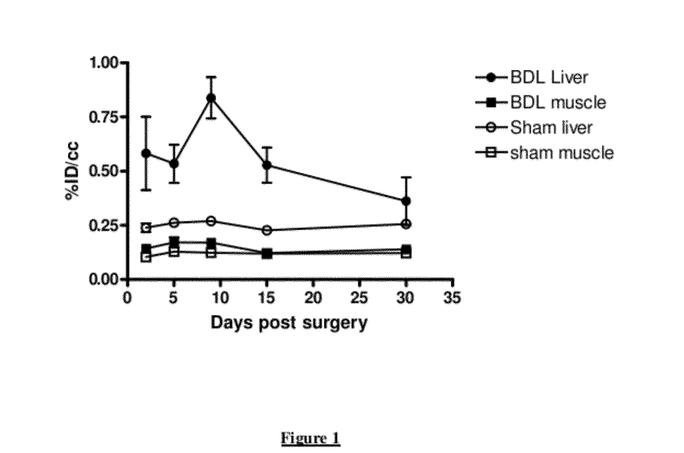

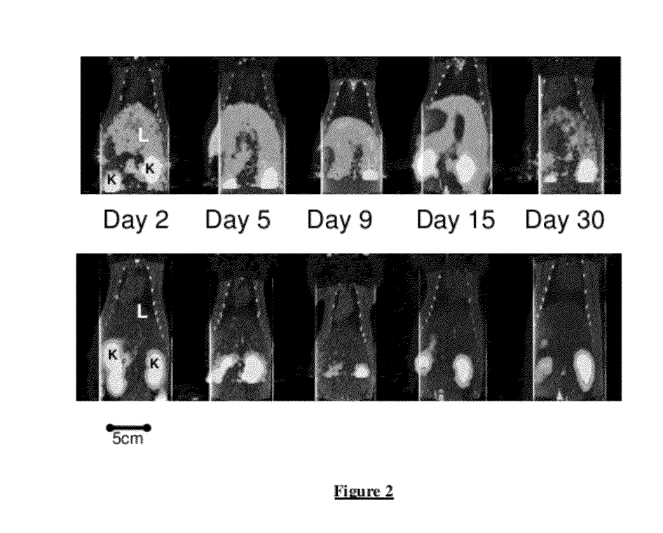

For assessment of PET Tracer 1 in the BDL rat model, static PET images were acquired longitudinally (imaged at 60-90 minutes post-injection) at days 2, 5, 9, 15 and 30 post bile duct ligation surgery or sham surgery. The PET images were co-registered with corresponding CT images.
Prior to the PET image commencing, the histogram and acquisition parameters were inputted into microPET Manager (software controlling data acquisition and processing). The reconstruction parameters were set as follows:
Fourier rebinning algorithm
2D Filter Back Projection with Ramp filter
Image zoom of 2
Scatter correction chosen
Raw image list mode data was saved in Intel/VAX-4-byte float format.
   Reconstructed data was saved in .img format (native)
   For the image acquisition the parameters were set as follows:
1800 second acquisition (static at 60-90 minutes post-injection)
1 bed position only
Energy windows set at 350-750 keV
Timing window set to 6 nSec
   For imaging studies, the information was additionally collected and stored in one list mode file. Images were recontructed as one static frame (1×30 minutes). Following reconstruction of the images ROI's were drawn on appropriate areas and activity/cm³ data generated using Amide software.
   Prior to the start of the imaging study, the anesthetised animal was fitted into the custom-made PET animal bed with fiducial markers attached. The animal was placed in the prone head first position fixed within the animal bed. The centre of the liver was lined up with the laser cross hairs, and the bed moved in the horizontal position into the camera by 100 mm. Data was analysed using Asipro and Amide software.
   Following completion of the PET imaging phase, the animal, still anaesthetised and affixed to the bed, was transferred to the CT camera. Without changing the position of the animal, the bed was positioned using the laser cross hairs in order that the thorax and abdomen of the animal was within the field of view. Using the microCAT II image acquisition software, the camera and CT scan parameters were set as follows:
Total rotations set at 360°
Total number of rotation steps set at 200
Total number of calibration exposures set at 25
Binning set at 4×4 (to give resolution of ˜200 μm)
Exposure time set at 400 ms
Camera set to 70 kVp voltage with 500 μA
   The acquired data was reconstructed using the image reconstruction, visualisation and analysis program (RVA2).
   The whole data set was reconstructed using Volume-3D (feldkamp cone beam) algorithm and with a Shepp-Logan filter applied. This enabled transaxial slices to be generated, viewed and stored as individual .CT files. A raw-3D dataset was stored along with the header file for further analysis in Amide.
   A significant difference in liver retention of PET Tracer 1 was shown between BDL and sham operated animals at days 2-15 post surgery (p<0.05), data shown in Table 1 and illustrated in FIGS. 1 and 2.
   The greatest difference between BDL and sham rat liver uptake was observed on day 9 with BDL liver uptake 0.84±0.10% ID/cc (n=3) compared to 0.27±0.02% ID/cc (n=3) in sham and on day 15 0.53±0.08% ID/cc (n=2) in BDL and 0.23±0.02% ID/cc (n=2) in sham was observed. By day 30 there was no significant difference in PET Tracer 1 uptake with 0.36±0.11% ID/cc (n=4) observed in BDL liver vs 0.26±0.02% ID/cc (n=4) in sham (data summarised in Table 1 below).

TABLE 1
Summary of % ID/cc in rat livers at different time point post-BDL
and sham surgery after 1 hour post i.v. injection of PET Tracer 1.

| Day post | BDL liver uptake | Sham liver uptake | |
|---|---|---|---|
| surgery | % ID/cc | % ID/cc | Significance |
| 2 | 0.58 ± 0.17 (n = 4) | 0.24 ± 0.03 (n = 3) | *p < 0.05 |
| 5 | 0.53 ± 0.09 (n = 4) | 0.26 ± 0.01 (n = 3) | **p < 0.01 |
| 9 | 0.84 ± 0.10 (n = 3) | 0.27 ± 0.02 (n = 3) | **p < 0.01 |
| 15 | 0.53 ± 0.08 (n = 2) | 0.23 ± 0.02 (n = 2) | *p < 0.05 |
| 30 | 0.36 ± 0.11 (n = 4) | 0.26 ± 0.02 (n = 3) | p > 0.05 |

In summary these data show that the uptake of PET Tracer 1 is significantly higher in BDL animal livers alone from 2 days post surgery suggesting that the BDL fibrosis model results in increased binding of the tracer PET Tracer 1 in the liver.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

1. A method to determine the presence, location, and/or amount of fibrogenic tissue in the liver of a subject, said method comprising the following steps:
(i) administering a detectable quantity of a positron emission tomography (PET) tracer of Formula I to said subject;
(ii) allowing the administered PET tracer of step (i) to bind to any fibrogenic tissue in said liver;
(iii) detecting signals emitted by the bound PET tracer of step (ii) by PET; and,
(iv) generating an image representative of the location and/or amount of said signals;
wherein said PET tracer is of Formula I: 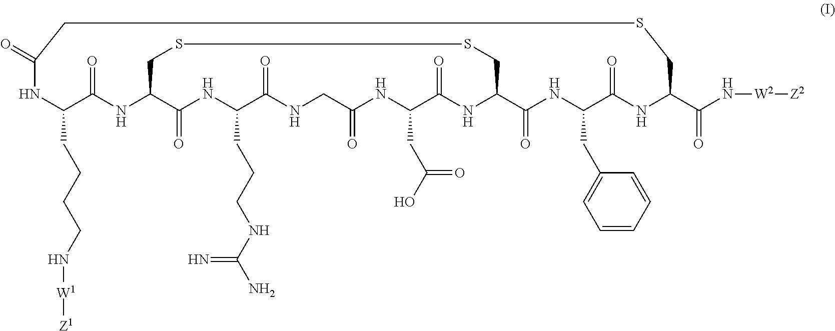
wherein:
one of Z¹ and Z² is a group comprising ¹⁸F, and the other of Z¹ and Z² is hydrogen; and, each of W¹ and W² is independently a bivalent linker moiety of either Formula Ia: 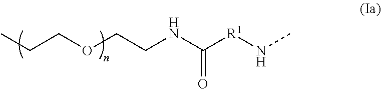
wherein
n is an integer from 1 to 10;
R¹ is C₁₋₅ alkylene, C₂₋₅ oxoalkylene, C₁₋₅ oxaalkylene, or is a C₂₋₅ carbonyl-substituted oxaalkylene; and,
the dotted line represents the point of attachment to either Z¹ or Z² or of Formula Ib: 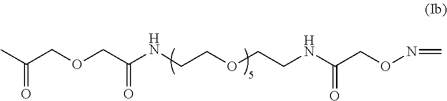
wherein the right hand double bond represents the point of attachment to either Z¹ or Z².

2. The method of claim 1, wherein said group comprising ¹⁸F of Formula I is [¹⁸F]fluorophenyl.

3. The method of claim 1, wherein Z¹ of Formula I is said group comprising ¹⁸F.

4. The method as defined in of claim 1, wherein n of Formula Ia is from 3 to 5.

5. The method of claim 1, wherein W¹ is a bivalent linker of Formula Ib.

6. The method of claim 1, wherein R¹ of Formula Ia is C₁₋₅ oxaalkylene.

7. The method claim 6, wherein R¹ is C₁₋₃ oxaalkylene.

8. The method of claim 7, wherein R¹ is —CH₂—O—.

9. The method of claim 1, wherein W² is a bivalent linker of Formula Ia wherein n is 3.

10. The method of claim 1, wherein R¹ of Formula Ia is C₂₋₅ carbonyl-substituted oxaalkylene.

11. The method of claim 10, wherein R¹ is C₂₋₄ carbonyl-substituted oxaalkylene.

12. The method of claim 11, wherein R¹ is —CH₂—O—CH₂—C(═O)—.

13. The method of claim 1, wherein said PET tracer of Formula I is provided as a radiopharmaceutical composition together with a biocompatible carrier in a form suitable for mammalian administration.

14. The method of claim 1, wherein said subject is an intact mammalian body in vivo.

15. The method of claim 1 which is carried out at two separate points in time.

16. The method of claim 15, wherein an antifibrogenic treatment is applied to said subject in between said two separate points in time.

17. The method of diagnosis of claim 1, further comprising the additional step of (v) attributing the location and/or amount of signals to a particular clinical picture.

18-19. (canceled)
